(19) United States
(12) Plant Patent Application Publication
(10) Pub. No.: US 2004/0268454 P1
(43) Pub. Date: Dec. 30, 2004

(54) Cineraria plant named Sunsenerapi

(76) Inventor: Kiyoshi Miyazaki, Hikone (JP)

Correspondence Address: CHRISTIE, PARKER & HALE, LLP, PO BOX 7068, PASADENA, CA, 91109-7068, US

(21) Appl. No.: 10/611,355

(22) Filed: Jun. 30, 2003

(51) Int. Cl. A01H 5/00

(52) U.S. Cl. PLT/263

(57) ABSTRACT

Disclosed herein is a Senecio genus plant named ‘Sunsenerapi’ being of a high height and dome-shaped plant with abundant branching. There are several pubescences on its stem. Petiole length is longer than in Senecio L. It has large flower cluster. Flowers are single flowered and have no marginal variegation. The color of the petal is light purplish pink and the disk flower is strong reddish purple. Blooming term is longer than Senecio cruentus. Flower buds grow one after another from axil.

### BOTANICAL/COMMERCIAL CLASSIFICATION

Senecio cruentus×Senecio heritieri/Cineraria Plant

### VARIETAL DENOMINATION

cv. ‘Sunsenerapi’

### BACKGROUND OF THE VARIETY

The present invention relates to a new and distinct variety of Senecio genus plant named ‘Sunsenerapi’. ‘Sunsenerapi’ is a distinct and unique variety, which is a dome-shaped plant of height with abundant branching, light purplish pink flowers, and a long blooming term.

There are many varieties in Senecio L. Senecio cruentus is well known as ‘Cineraria’ and cultivated in the world. There are many cultivated varieties with flowers of a single color of white, pink, red, blue or violet. Some varieties have marginal variegation with off color parts.

The female parent used in the crossing of ‘Sunsenerapi’ is a clone of our own breeding line, Senecio cruentus, ‘8S-84e’ (not patented in the United States), which is a compact, dome-shaped plant, 16 cm in height. The stems are thick, 8.0 mm in diameter, with no anthocyanin coloration. The leaf is of a serrated heart form and moderate yellow green. The leaf size is medium, 12.0 cm long, 12.5 cm wide. The flower is single flowered and has white petals with white disk flowers having no marginal variegation. ‘8S-84e’ has some scent.

The pollen parent used in the crossing of ‘Sunsenerapi’ is our breeding line, Senecio heritieri (not patented or sold in the United States), which was introduced from England at first. Senecio heritieri is a high and dome-shaped plant, 26 cm in height with abundant branching. Stems are 5.1 mm in diameter, with no anthocyanin coloration. The leaf is in a serrated heart form and light yellowish green. The leaf size is small, 5.5 cm long, 6.3 cm wide. The flower is single flowered, having strong purple petals with vague white parts and strong reddish purple disk flowers. Senecio heritieri has no scent.

Progress

The controlled crossing with Senecio cruentus ‘8S-84e’ and Senecio heritieri was conducted at Omori-cho, yokaichi-shi, Shiga, Japan in February, 1999. Seedlings from this crossing were grown since September, 1999. Four strains were selected in January 2000 among them in view of flower color and earliness. After multiplication by tissue culture, the botanical characteristics of the selected new strains were tested in pots since September 2000, using parent varieties and ‘Sunsenere’ (patented in the United States: U.S. Pat. No. PLANT 12162, at 23.Oct.2001) and ‘Midget’ (not patented in the United States) for comparison, since 2000. As a result, one strain was selected and it is confirmed that the selected variety met our criteria of being uniform and stable in its characteristics.

Then this new variety of Senecio genus plant was named ‘Sunsenerapi’ (Senecio cruentus×Senecio heritieri)

Senecio cruentus, ‘8S-84e’, and Senecio heritieri are presently maintained at the Omori-cho, Yokaichi-shi, Shiga-ken, Japan.

In the following descriptions, the color-coding is in accordance with the Horticultural Colour Chart of The Royal Horticultural Society, London, England (R.H.S. Colour Chart).

The botanical characteristics of the female parent plant Senecio cruentus, ‘8S-84e’, used in the crossing of ‘Sunsenerapi’ are as follows.

Plant:

Growth habit.—Dwarf compact.

Height.—16 cm.

Stem:

Thickness.—8.0 mm.

Color.—Moderate yellowish green (R.H.S.C.C. No.139C).

Anthocyanin coloration.—Absent.

Branching.—Fair.

Pubescence.—Sparse.

Length of third internode below flag leaf.—0.5 cm.

Leaf:

Whole shape.—Heart form with a swollen basal part, which is notched at where the basal part of the blade attaches to the petiole.

Depth of concavity of leaf margin.—Medium.

Type of convexity.—Acute.

Apex shape.—obtuse.

Base shape.—Cordate.

Degree of Undulation.—Fair.

Length.—12.0 cm.

Width.—12.5 cm.

Diameter of petiole.—5.0 mm.

Length of petiole.—5.5 cm.

Color of upper surface.—Moderate yellow green (R.H.S.C.C. No.137C).

Color of reverse surface.—Grayish yellow green (R.H.S.C.C. No.138B).

Anthocyanin coloration of reverse surface.—Absent.

Pubescence of upper surface.—Present.

Pubescence of reverse surface.—Dense.

Color of pubescence of reverse surface.—White.

Flower (single flowered):

Shape of flower cluster.—Flat.

Diameter of flower cluster.—20 cm.

Height of flower cluster.—8 cm.

Transected shape of corolla.—Flat.

Diameter of flower.—5.5 cm.

Size of disk flower.—1.2 cm.

Color of petal.—White (R.H.S.C.C. No.155D).

Marginal variegation.—Absent.

Color of disk flower.—White (R.H.S.C.C. No.155D).

Petal length.—2.1 cm.

Petal width.—1.0 cm.

Shape of petal.—Elliptical.

Lengthwise warp of petal.—Flat.

Concavity of patal tip.—Present.

Shape of petal tip.—Rounded.

Number of ray flower 13.

Number of disk flower.—105.

Diameter of pedicel of the first flower.—1.6 mm.

Length of pedicel of the first flower.—2.4 cm.

Number of flowers per a flower cluster.—75.

Scent.—Present.

Calyx:

Degree of concavity.—Flat.

Degree of recurvature.—2.2 mm.

Color.—Moderate,yellow green (R.H.S.C.C. No.139C).

Anthocyanin coloration.—Absent.

Pistil:

Color.—Light yellow (R.H.S.C.C. No.11B).

Number.—1.

Type.—Style branches truncate (i.e. the top of the style is separated into two and the shape of the top is truncated).

Stamen:

Color.—Brilliant yellow (R.H.S.C.C. No.12B).

Type.—5 anthers are connate, with separated filaments.

Blooming period.—January (Sowing in August).

Hardiness:

Cold.—Good.

Rain.—Good.

Heat.—Good.

Resistance:

Disease.—Good.

Insect.—Good.

The botanical characteristics of the male parent plant Senecio heritieri used in the crossing of ‘Sunsenerapi’ are as follows.

Plant:

Growth habit.—Dwarf compact.

Height.—26 cm.

Stem:

Thickness.—5.1 mm.

Color.—Very pale green (R.H.S.C.C. No.128D).

Anthocyanin coloration.—Absent.

Branching.—Abundunt.

Type of primary lateral shoot.—Branch from every node.

Pubescence.—Dense.

Length of third internode below flag leaf.—0.8 cm.

Leaf:

Whole shape.—Heart form with a swollen basal part, which is notched at where the basal part of the blade attaches to the petiole.

Depth of concavity of leaf margin.—Medium.

Type of convexity.—Acute.

Apex shape.—Obtuse.

Base shape.—Cordate.

Degree of Undulation.—Weak.

Length.—5.5 cm.

Width.—6.3 cm.

Diameter of petiole.—4.2 mm.

Length of petiole.—7.5 cm.

Color of upper surface.—Light yellowish green (R.H.S.C.C. No.136D).

Color of reverse surface.—Very pale green (R.H.S.C.C. No.128D).

Anthocyanin coloration of reverse surface.—Absent.

Pubescence of upper surface.—Dense.

Pubescence of reverse surface.—Dense.

Color of pubescence of reverse surface.—White.

Flower (single flowered):

Shape of flower cluster.—Uneven.

Diameter of flower cluster.—18 cm.

Height of flower cluster.—15 cm.

Transected shape of corolla.—Flat.

Diameter of flower.—4.8 cm.

Size of disk flower.—0.9 cm.

Color of petal.—Strong purple (R.H.S.C.C. No.81B).

Marginal variegation.—Present.

Diameter of off color part.—1.3 cm.

Border of marginal variegation.—Vague.

Color of disk flower.—Strong reddish purple (R.H.S.C.C. No.72A).

Petal length.—2.2 cm.

Petal width.—0.4 cm.

Shape of petal.—Rectangular.

Lengthwise warp of petal.—Flat.

Concavity of patal tip.—Present.

Shape of petal tip.—Acute.

Number of ray flower.—13.

Number of disk flower.—110.

Diameter of pedicel of the first flower.—1.2 mm.

Length of pedicel of the first flower.—2.5 cm.

Number of flowers per a flower cluster.—35.

Scent.—Absent.

Calyx:

Degree of concavity.—Flat.

Degree of recurvature.—3.1 mm.

Color.—Very pale green (R.H.S.C.C. No.128D).

Anthocyanin coloration.—Present.

Pistil:

Color.—Strong reddish purple (R.H.S.C.C. No.72A).

Number.—1.

Type.—Style branches truncate (i.e. the top of the style is separated into two and the shape of the top is truncated).

Stamen:

Color.—Strong reddish purple (R.H.S.C.C. No.72A).

Type.—5 anthers are connate, with separated filaments.

Blooming period.—End of January (Sowing in August).

Hardiness:

Cold.—Good.

Rain.—Good.

Heat.—Good.

Resistance:

Disease.—Good.

Insect.—Good.

The botanical characteristics of a similar variety ‘Sunsenere’ (Senecio cruentus×Senecio heritieri) which was patented in the United States, used for examination as a comparison variety are as follows.

Plant:

Growth habit.—Semi-dwarf erect.

Height.—25 cm.

Stem:

Thickness.—5.0 mm.

Color.—Moderate yellowish green (R.H.S.C.C. No.139C).

Anthocyanin coloration.—Present in parts of stem, e.g. can be seen at the part of peduncle.

Degree of anthocyanin coloration.—It is light grayish olive (R.H.S.C.C. No.197A) at the part wherein anthocyanin is present, but it is moderate yellowish green (R.H.S.C.C. No.138C) at the part where anthocyanin is absent.

Branching.—Abundant, i.e. 9 to 11 branches.

Type of primary lateral shoot.—Branch from every node.

Pubescence.—Sparse.

Length of third internode below flag leaf.—1.0 cm.

Leaf:

Whole shape.—Heart form with a swollen basal part, which is notched at where the basal part of the blade attaches to the petiole.

Depth of concavity of leaf margin.—The maximum depth of concavity measured from the average convexity peak height is 7 mm.

Type of convexity.—Acute.

Apex shape.—Obtuse.

Base shape.—Cordate.

Degree of Undulation.—Weak.

Length.—8.6 cm.

Width.—10.3 cm.

Diameter of petiole.—3.2 mm.

Length of petiole.—9.0 cm.

Color of upper surface.—Moderate yellow green (R.H.S.C.C. No.137C).

Color of reverse surface.—Moderate yellow green (R.H.S.C.C. No.138C).

Anthocyanin coloration of reverse surface.—Absent.

Pubescence of upper surface.—Dense.

Pubescence of reverse surface.—Dense.

Color of pubescence of reverse surface.—White.

Flower (single flowered):

Shape of flower cluster.—Uneven.

Diameter of flower cluster.—34 cm.

Height of flower cluster.—22 cm.

Transected shape of corolla.—Flat.

Diameter of flower.—6.4 cm.

Size of disk flower.—1.3 cm.

Color of petal.—Vivid reddish purple (R.H.S.C.C. No.78A).

Marginal variegation.—Absent.

Color of disk flower.—Deep reddish purple (R.H.S.C.C. No.80A).

Petal length.—2.6 cm.

Petal width.—0.8 cm.

Shape of petal.—Rectangular.

Lengthwise warp of petal.—Flat.

Concavity of patal tip.—Present.

Shape of petal tip.—Acute.

Number of ray flower.—15-16.

Number of disk flower.—130.

Diameter of pedicel of the first flower.—1.2 mm.

Length of pedicel of the first flower.—3.3 cm.

Number of flowers per a flower cluster.—88.

Scent.—Present.

Calyx:

Degree of concavity.—Flat.

Degree of recurvature.—1.2 mm.

Color.—Very pale green (R.H.S.C.C. No.130D).

Anthocyanin coloration.—Absent.

Pistil:

Color.—Vivid reddish purple (R.H.S.C.C. No.80A).

Number.—1.

Type.—Style branches truncate (i.e. the top of the style is separated into two and the shape of the top is truncated).

Stamen:

Color.—Vivid reddish purple (R.H.S.C.C. No.80A).

Type.—5 anthers are connate, with separated filaments.

Blooming period.—Start at the end of November or early December (cutting in July).

Hardiness:

Cold.—Good.

Rain.—Good.

Heat.—Good.

Resistance:

Disease.—Good.

Insect.—Good.

The botanical characteristics of a similar variety ‘Midget’, used for examination as a comparison variety are as follows.

Plant:

Growth habit.—Dwarf.

Height.—14 cm.

Stem:

Thickness.—5.6 mm.

Color.—Moderate yellow green (R.H.S.C.C. No.139C).

Anthocyanin coloration.—Present.

Degree of anthocyanin coloration.—Medium.

Branching.—Fair.

Pubescence.—Dense.

Length of third internode below flag leaf.—0.5 cm.

Leaf:

Whole shape.—Heart form with a swollen basal part, which is notched at where the basal part of the blade attaches to the petiole.

Depth of concavity of leaf margin.—Medium.

Type of convexity.—Acute.

Apex shape.—Acute.

Base shape.—Cordate.

Degree of Undulation.—Fair.

Length.—13.9 cm.

Width.—10.3 cm.

Diameter of petiole.—4.2 mm.

Length of petiole.—4.7 cm.

Color of upper surface.—Grayish yellow green (R.H.S.C.C. No.138A).

Color of reverse surface.—Grayish yellow green (R.H.S.C.C. No.138B).

Anthocyanin coloration of reverse surface.—Absent.

Pubescence of upper surface.—Present.

Pubescence of reverse surface.—Dense.

Color of pubescence of reverse surface.—White.

Flower (single flowered):

Shape of flower cluster.—Flat.

Diameter of flower. cluster.—22 cm.

Height of flower cluster.—8 cm.

Transected shape of corolla.—Fairly closed.

Diameter of flower.—3.2 cm.

Size of disk flower.—0.8 cm.

Color of petal.—Vivid reddish purple (R.H.S.C.C. No.74A).

Marginal variegation.—Absent.

Color of disk flower.—Strong reddish purple (R.H.S.C.C. No.72A).

Petal length.—1.5 cm.

Petal width.—0.8 cm.

Shape of petal.—Elliptical.

Lengthwise warp of petal.—Flat.

Concavity of patal tip.—Present.

Shape of petal tip.—Acute.

Number of ray flower.—13.

Number of disk flower.—86.

Diameter of pedicel of the first flower.—1.5 mm.

Length of pedicel of the first flower.—2.5 cm.

Number of flowers per a flower cluster.—180.

Scent.—Present.

Calyx:

Degree of concavity.—Flat.

Degree of recurvature.—2.2 mm.

Color.—Moderate yellow green (R.H.S.C.C. No.139C).

Anthocyanin coloration.—Absent.

Pistil:

Color.—Strong reddish purple (R.H.S.C.C. No.72A).

Number.—1.

Type.—Style branches truncate (i.e. the top of the style is separated into two and the shape of the top is truncated).

Stamen:

Color.—Strong reddish purple (R.H.S.C.C. No.72A).

Type.—5 anthers are connate, with separated filaments.

Blooming period.—January (Sowing in August).

Hardiness:

Cold.—Good.

Rain.—Good.

Heat.—Good.

Resistance:

Disease.—Good.

Insect.—Good.

### SUMMARY OF THE VARIETY

‘Sunsenerapi’ is a dome-shaped plant of height (average height of 31.8 cm in the blooming period). There is a branch from every node and branching is abundant, i.e. 9 to 11 branches. The stem is 4.8 mm in diameter with few pubescences.

The leaves are small, 13.9 cm long, 9.7 cm wide. The leaves are shaped in a serrated heart form with a swollen basal part, which is notched at where the basal part of the blade attaches to the petiole. The leaf color is moderate olive green, with no anthocyanin coloration on the reverse side. Pubescence on both sides of leaf is dense. The petiole is 10.0 cm long and 3.4 mm in diameter.

The new variety has large flower cluster which are high and uneven in shape (i.e. the locations of the individual flowers forming the cluster are uneven or not flat). A flower cluster means a gathering of heads in a plant of the new variety. A head is a flower consisting of a disk and a plurality of petals, i.e. ray flowers. The flower is single flowered with no marginal variegation. The corolla has a flat transected shape (i.e. when the corolla opens, petals are flat when viewed from the side). The flower is 6.7 cm in diameter, while the disk flower is 1.3 cm in diameter. The petals is 3.1 cm long and 0.8 cm wide. The color of the petal is light purplish pink (R.H.S.C.C. No.75C), while the color of the disk is strong reddish purple (R.H.S.C.C. No.77B). One flower has 13 ray flowers and 167 disk flowers. A ray flower has a pistil, but no stamen. A disk flower is tubular and has a pistil and a stamen formed by 5 connate anthers with separated filaments. 155 flowers are in a flower cluster. The degree of recurvature of calyx is 2.0 mm. The calyx is flat and has an anthocyanin coloration. Pedicel is 3-9 cm long and 1.2 mm in diameter. The flowers have some scent.

Blooming period is medium and blooming term is long. After cutting in July, flowers start to bloom at the end of the January in Omori-cho, Yokaichi-shi, Shiga-ken, Japan. The blooming continues from January to May, if under appropriate control. Around a temperature of 15° C., the individual bloom lasts two weeks. The flower buds grow one after another from the axil. The flower is not self-cleaning because petals remain attached in a wilted and dry state as the flower fully matures.

The new variety has moderate cold hardiness and resistance to heat. The new variety does not die at around 0° C., but when frost, occurs the cells can be necrosed resulting in the death of the plant. Usually, Senecio cruentus has a tendency of slightly reduced growth in a hot season, whereas the new variety has no problem in growing in the hot season.

The fertility of the new variety is low. Generally, Senecio genus plants have high fertility (i.e. bear many seeds per flower). In contrast, ‘Sunsenerapi’ bears no seed or very few seeds per flower. When no seed is formed, there is a part corresponding to a seed coat, which remains in an immature state, i.e. without embryo and endosperm inside the immature seed coat.

The new variety, ‘Sunsenerapi’, differs from similar varieties, ‘Sunsenere’ and ‘Midget’, of the Senecio genus and from parent varieties Senecio heritieri and ‘8S-84e’ in the following points.

1. ‘Sunsenerapi’ is a high type plant, 31.8 cm in height. ‘Sunsenere’ is a high type plant, 25 cm in height. ‘Midget’ is a compact type plant, 14 cm in height. The pollen parent Senecio heritieri is a semi-compact type plant, 26 cm in height and female plant ‘8S-84e’ is a compact type plant, 16 cm in height.

2. The flower of ‘Sunsenerapi’ has light purplish pink petals, strong reddish purple disk flowers and no marginal variegation. That of ‘Sunsenere’ has vivid reddish purple petals, deep reddish purple disk flowers and no marginal variegation. That of ‘Midget’ has vivid reddish purple petals, strong reddish purple disk flowers and no marginal variegation. That of female parent ‘8S-84e’ has white petals, white disk and no marginal variegation. That of pollen parent Seneclo heritieri has strong purple petals, strong reddish-purple disk flowers and white marginal part.

3. The petiole length of ‘Sunsenerapi’ is longer than that of ‘Sunsenere’ or ‘Midget’.

4. ‘Sunsenerapi’ has more pubescence of stems than that of ‘Sunsenere’ and fewer than that of ‘Midget’.

5. The blooming term of ‘Sunsenerapi’ is longer than that of ‘Midget’.

6. The fertility of ‘Sunsenerapi’ is lower than that of ‘Midget’.

The plant height and flower color are most distinctive characteristics of this new variety, ‘Sunsenerapi’.

### BRIEF DESCRIPTION OF THE DRAWING

FIG. 1 is a photograph giving a partial view of the new variety of Senecio genus plant named ‘Sunsenerapi’ planted in a pot.

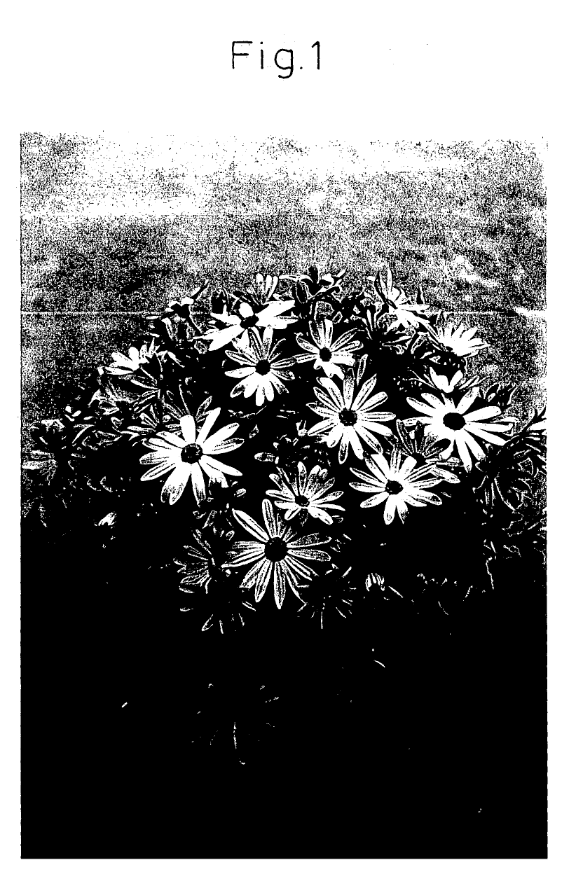

FIG. 2 is a photograph of flowers of the new variety of Senecio genus plant named ‘Sunsenerapi’.

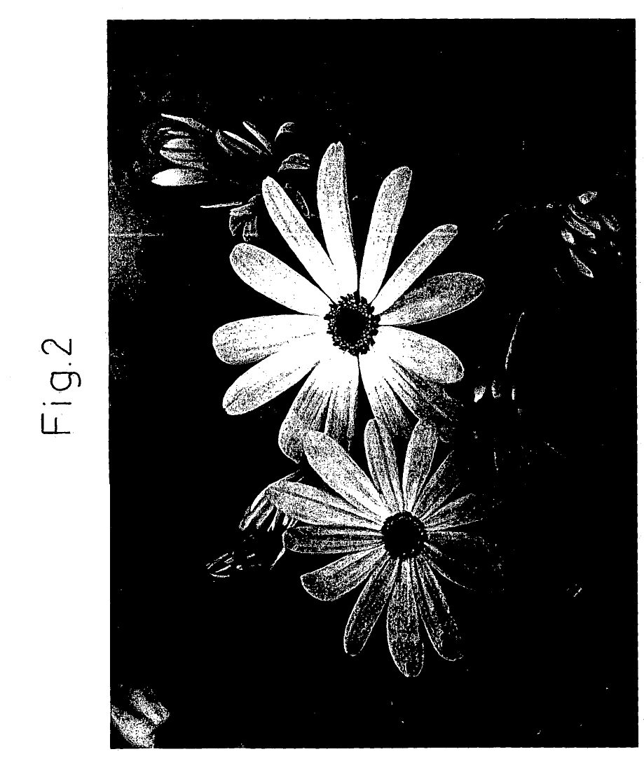

### DESCRIPTION OF THE NEW VARIETY

The botanical characteristics of this new and distinct variety of Senecio genus plant named ‘Sunsenerapi’ 7 months after cutting in July are as follows (i.e. these botanical characteristics were observed in January of the year following the cutting with the new variety planted in a 15 cm pot and grown in a greenhouse, the lowest temperature of which was controlled at 12° C. in winter).

Plant:

Growth habit.—Semi-dwarf erect.

Height.—31.8 cm.

Spread.—35 cm.

Stem:

Thickness.—4.8 mm.

Color.—Very pale green (R.H.S.C.C. No.130D).

Anthocyanin coloration.—Absent.

Branching.—Abundant, i.e. 9 to 11 branches.

Type of primary lateral shoot.—Branch from every node.

Pubescence.—Medium.

Length of third internode below flag leaf.—1.8 cm.

Leaf:

Whole shape.—Heart form with a swollen basal part, which is notched at where the basal part of the blade attaches to the petiole.

Depth of concavity of leaf margin.—The maximum depth of concavity measured from the average convexity peak height is 5 mm.

Type of convexity.—Acute.

Apex shape.—Acute.

Base shape.—Cordate.

Degree of undulation.—Weak.

Length.—13.9 cm.

Width.—9.7 cm.

Diameter of petiole.—3.4 mm.

Length of petiole.—10.0 cm.

Color of upper surface.—Moderate olive green (R.H.S.C.C. No.146A).

Color of reverse surface.—Moderate yellow green (R.H.S.C.C. No.147D).

Anthocyanin coloration of reverse surface.—Absent.

Pubescence of upper surface.—Sparse.

Pubescence of reverse surface.—Dense.

Color of pubescence of reverse surface.—White.

Flower (single flowered):

Shape of flower cluster.—Round.

Diameter of flower cluster.—34 cm.

Height of flower cluster.—22 cm.

Transected shape of corolla.—Flat.

Diameter of flower.—6.7 cm.

Size of disk flower.—1.3 cm.

Color of petal.—Light purplish pink (R.H.S.C.C. No.75C).

Marginal variegation.—Absent.

Color of disk flower.—Strong reddish purple (R.H.S.C.C. No.77B).

Petal length.—3.1 cm.

Petal width.—0.8 cm.

Shape of petal.—Long oblong.

Lengthwise warp of petal.—Flat.

Concavity of patal tip.—Present.

Shape of petal tip.—Acute.

Number of ray flower.—13.

Number of disk flower.—167.

Diameter of pedicel of the first flower.—1.2 mm.

Length of pedicel of the first flower.—3.9 cm.

Number of flowers per a flower cluster.—155.

Scent.—Present.

Calyx:

Degree of concavity.—Flat.

Degree of recurvature.—2.0 mm.

Color.—Moderate yellow-green (R.H.S.C.C. No.139C).

Anthocyanin coloration.—Absent.

Pistil:

Color.—Vivid purple (R.H.S.C.C. No.82A).

Number.—1.

Type.—Style branches truncate (i.e. the top of the style is separated into two and the shape of the top is truncated).

Stamen:

Color.—Dark yellow (R.H.S.C.C. No.153B).

Type.—5 anthers are connate, with separated filaments.

Blooming period.—Start at the end of January (cutting in July).

Hardiness:

Cold.—Good.

Rain.—Good.

Heat.—Good.

Resistance:

Disease.—Good.

Insect.—Good.

The new variety and Senecio cruentus have similar resistance to powdery mildew, leaf spot, aphid, whitefly, and thrips. The new variety, ‘Sunsenerapi’, is a tall type plant and most suitable for flower potting.

This new and distinct variety of Senecio genus plant named ‘Sunsenerapi’ was asexually reproduced by cutting at Omori-cho, Yokaichi-shi, Shiga-ken, Japan and the homogeneity and stability thereof were confirmed.

1. A new and distinct variety of Senecio genus plant named ‘Sunsenerapi’, substantially as herein illustrated and described.
